(19) United States
(12) Patent Application Publication
(10) Pub. No.: US 2023/0263228 A1
(43) Pub. Date: Aug. 24, 2023

(54) Inverted Heater

(71) Applicant: JT International SA, Geneva (CH)

(72) Inventor: Peter Loveday, Epsom (GB)

(73) Assignee: JT International SA, Geneva (CH)

(21) Appl. No.: 18/020,326

(22) PCT Filed: Aug. 10, 2021
(86) PCT No.: PCT/EP2021/072235

§ 371 Date: Feb. 8, 2023

(30) Foreign Application Priority Data
Aug. 14, 2020 (EP) 20191171.6

(51) Int. Cl. A24F 40/46; A24F 40/42; A24F 40/10; A24F 40/44

(52) U.S. Cl. CPC A24F 40/46; A24F 40/42; A24F 40/10; A24F 40/44

(57) ABSTRACT

A capsule has a first end to engage with an electronic cigarette device and a second end arranged as a mouthpiece portion having a vapor outlet, a liquid reservoir located towards the first end and configured to store a liquid to be vaporised; a vaporising chamber located towards the second end and having an air inlet and a vapor outlet; a heating element arranged between the liquid reservoir and the vaporising chamber and configured to vaporise liquid received from the liquid reservoir and generate a vapor in the vaporising chamber; and a flow path extending between the vaporising chamber and the mouthpiece to allow the generated vapor to flow from the vaporising chamber to the mouthpiece. The heating element includes: a heating surface exposed to the vaporising chamber, at a first end of the heating element and a capillary element having a capillary surface exposed to the liquid reservoir.

### FIELD OF INVENTION

The present invention relates to a heater in a capsule for an electronic cigarette.

### BACKGROUND

Electronic cigarettes are an alternative to conventional cigarettes. Instead of generating a combustion smoke, they vaporize a liquid which can be inhaled by a user. The liquid typically comprises an aerosol-forming substance, such as glycerin or propylene glycol that creates the vapor. Other common substances in the liquid are nicotine and various flavorings.

The electronic cigarette is a hand-held inhaler system, comprising a mouthpiece section, a liquid store, and a power supply unit. Vaporization is achieved by a vaporizer or heater unit which typically comprises a heating element in the form of a heating coil and a fluid transfer element, such as a wick, arranged to transfer fluid from the liquid store to the heating element. Vaporization occurs when the heater heats up the liquid in the fluid transfer element until the liquid is transformed into vapor. The vapor can then be inhaled via an air outlet in the mouthpiece.

The electronic cigarette may comprise a capsule seating which is configured to receive disposable consumables in the form of capsules. Capsules comprising the liquid store and the vaporizer are often referred to as “cartomizers”. In this case, the vaporizer of the cartomizer is connected to the power supply unit when received in the capsule seating such that electricity can be supplied to the heater of the cartomizer to heat the liquid to generate the vapor. Often some form of mechanical mechanism is used to retain the capsule in the capsule seating such that it does not fall out and separate from the device.

In order to transfer liquid from the liquid store to the heating element, the wick must be arranged between the liquid store and vaporization chamber such that, when the wick is heated, capillary action transports liquid through the porous structure of the wick from the liquid store to the heating element.

It is an object of the present invention to provide an improved heater for an electronic cigarette.

### SUMMARY OF INVENTION

According to a first aspect there is provided a capsule for an electronic cigarette, the capsule having a first end configured to engage with an electronic cigarette device and a second end arranged as a mouthpiece portion having a vapor outlet. The capsule further comprises a liquid reservoir located towards the first end of the capsule, the liquid reservoir configured to store a liquid to be vaporised. A vaporising chamber is located towards the second end of the capsule, the vaporising chamber comprising an air inlet and a vapor outlet. A heating element is arranged between the liquid reservoir and the vaporising chamber, the heating element configured to vaporise liquid received from the liquid reservoir and generate a vapor in the vaporising chamber. A flow path extends between the vaporising chamber and the mouthpiece to allow the generated vapor to flow from the vaporising chamber to the mouthpiece. The heating element further comprises a heating surface at a first end of the heating element and exposed to the vaporizing chamber and a capillary element comprising a capillary surface exposed to the liquid reservoir.

Preferably, the capillary surface is located at a second end of the heating element. In some examples, the heating surface may be substantially planar. By arranging the heating surface at an end of the heating element, and preferably the heating surface being substantially planar, liquid is more evenly distributed to the heating surface from the liquid reservoir.

Preferably, at the second end of the heating element, the capillary surface extends at least partially parallel to the heating surface.

The heating surface may be arranged to provide fluid communication between the heating element and the vaporising chamber. The capillary element, in particular the capillary surface, may be arranged to provide fluid communication between the heating element and the liquid reservoir. This arrangement means that the heating element has an inverted configuration compared to known heating elements. It should be noted that the term inverted is used with reference to the capsule when held in its operative configuration.

In other words, the inverted orientation of the heating element means that air flows across an upper surface of the heating element and the liquid to be vaporised is fed from a lower surface of the heating element. In conventional heating elements, however, liquid is fed to the heating element from above the heating element or from a side of the heating element. Leakage of liquid into the flow path is reduced, because the air flow path is located above the heating element. Advantageously, the arrangement of the present invention ensures that any fluid that has leaked is able to flow back onto or into the heating element rather than onto other components of the capsule. The leaked fluid can therefore be automatically vaporised by the heating element, without the need to redirect the leaked fluid back to the heating element. In other words, the inverted heating element means that liquid travels through the heating element, from the liquid reservoir, against the force of gravity when the capsule is held in its operative configuration, which reduces the chance of liquid leaking into the capsule from the liquid reservoir. Additionally, the capsule described herein provides a simplified construction with fewer parts, and reduced assembly stages.

The heating element may comprise a capillary-type heating element. This may facilitate efficient delivery of liquid from the liquid reservoir to the vaporising chamber via capillary action.

In some cases, the heating surface of the heating element comprises an electrically resistive surface. Preferably, the heating surface of the heating element comprises a porous material. The electrically resistive surface may be attached, preferably printed, to a porous material. The porous material may comprise a rigid ceramic. The porous material may facilitate transfer of liquid through the heating element received from the liquid reservoir. The electrically resistive surface may ensure that only a portion of the heating element is arranged to vaporize the liquid received from the liquid reservoir.

The liquid reservoir may comprise a buffer reservoir. Preferably, the buffer reservoir is in fluid communication with a storage reservoir via a conduit. The storage reservoir may be arranged to store an additional volume of liquid to be vaporized. The conduit therefore enables fluid to flow between the storage reservoir and the buffer reservoir.

The storage reservoir may be located between the second end of the capsule and the heating element. Preferably, the storage reservoir is located closer to the mouthpiece than the buffer reservoir. This arrangement means that the storage reservoir is preferably located above the buffer reservoir when the capsule is held vertically in its operative configuration such that liquid can flow from the storage reservoir to the buffer reservoir via the conduit under the action of gravity. The buffer reservoir can therefore be filled up, or replenished, using liquid from the storage reservoir under the action of gravity alone, without the need for additional components. This reduces the complexity of the capsule.

The storage reservoir may be arranged above the heating surface of the heating element and the capillary surface may be arranged below the heating surface or at a second end of the heating element substantially opposite the heating surface, when the device is held vertically in its operative configuration. This configuration may allow fluid to be drawn into the heating element by the effect of capillarity against the force of gravity. This arrangement may provide a more even distribution of liquid to the heating element. The heating surface of the heating element may be substantially opposite to the liquid loading surface i.e. the capillary element. This may prevent dry burn of the heating element which is not desirable.

In some examples, at least part of the heating element is located within the buffer reservoir. In particular, the capillary surface of the capillary element may be located in the buffer reservoir. This ensures good fluid communication between the buffer reservoir and the heating element.

The heating element may comprise at least one gripping mechanism. The at least one gripping mechanism may be arranged to maintain the position of the heating element within the buffer reservoir. This may prevent the heating element from moving within the buffer reservoir, which may cause liquid within the buffer reservoir to leak. Further, this may ensure that good fluid communication between the heating element and the buffer reservoir is maintained.

Preferably, the at least one gripping mechanism comprises at least one resiliently biased contactor. This may provide a secure gripping mechanism ensuring that the heating element remains in position, regardless of the orientation of the capsule. In particular, the resiliently biased gripping mechanism ensures that the heating element remains in contact with the buffer reservoir, even when the capsule is held upside down.

In some cases, the contactor comprises at least one spring blade extending from the gripping mechanism and arranged to engage the heating surface of the heating element. This provides a simple and convenient means of holding the heating element in place.

The buffer reservoir may comprise a substantially constant volume of liquid along the length of the buffer reservoir. Preferably the length of the buffer reservoir is adjacent at least one surface of the heating element. This arrangement may provide an increased contact surface area between the heating element and the buffer reservoir, ensuring optimal transfer of liquid from the buffer reservoir to the heating element.

Preferably, the heating element is supported in the buffer reservoir by at least one spacer. This may ensure that a space, or gap, is created between the heating element and a floor of the buffer reservoir to ensure that liquid is able to flow around the heating element. In some examples, the at least one spacer may comprise at least one rib. Alternatively, the at least one spacer may comprise at least one bump.

The at least one spacer may comprise part of the heating element. This may reduce the number of individual components within the capsule.

Preferably, the at least one spacer comprises a wall of the buffer reservoir. More preferably, the wall may be located substantially opposite to the heating element.

The buffer reservoir may be delimited by a holder of the capsule and a seal member. The holder may comprise a sump. The sump may be arranged to collect liquid which has not been vaporized and recycle this liquid back to the heating element.

The heating element may be located between the holder and a single seal member. Preferably, the seal member forms part of the vaporizing chamber, the air inlet, and the vapor outlet. The number of individual components within the capsule may therefore be reduced. Furthermore, by forming the vaporizing chamber, the inlet, and the vapor outlet, from a single component, namely the seal member, the likelihood of liquid leaking between adjoining components is reduced.

In some arrangements, the heating surface of the heating element may correspond to a surface of the seal member such that the heating surface of the heating element delimits the vaporizing chamber and forms a fluid seal. This may prevent the flow of vapor and liquid from the vaporizing chamber to the heating element.

In some arrangements, the heating element and seal member may separate the buffer reservoir and the vaporizing chamber. This configuration may help prevent the flow of liquid from the vaporizing chamber to the buffer reservoir.

According to another aspect there may be provided an electronic cigarette comprising a main body and a capsule wherein the main body comprises a power supply unit, electrical circuitry, and a capsule seating configured to connect with the capsule, the capsule comprising: a first end configured to engage with the electronic cigarette device and a second end arranged as a mouthpiece portion having a vapor outlet, the capsule further comprising: a liquid reservoir located towards the first end of the capsule, the liquid reservoir configured to store a liquid to be vaporised; a vaporising chamber located towards the second end of the capsule, the vaporising chamber comprising an air inlet and a vapor outlet; a heating element arranged between the liquid reservoir and the vaporising chamber, the heating element configured to vaporise liquid received from the liquid reservoir and generate a vapor; a flow path extending between the vaporising chamber and the mouthpiece to allow the generated vapor to flow from the vaporising chamber to the mouthpiece; wherein the heating element comprises: a heating surface at a first end of the heating element, the heating surface exposed to the vaporising chamber; and a capillary element comprising a capillary surface, the capillary surface exposed to the liquid reservoir. There may also be provided an electronic cigarette comprising a capsule according to any of the above described capsules.

### BRIEF DESCRIPTION OF DRAWINGS

Embodiments of the present invention will now be described by wait of example with reference to the accompanying drawings in which:

FIG. 1a shows a perspective view of part of a capsule for an electronic cigarette;

FIG. 1b shows an exploded perspective view of a capsule for an electronic cigarette;

FIG. 1c shows a perspective view of a capsule for an electronic cigarette;

FIG. 2a shows a perspective view of a seal member of a capsule for an electronic cigarette;

FIG. 2b shows a perspective view of a holder of a capsule for an electronic cigarette;

FIG. 3a shows an exploded perspective view of a lower housing portion of a capsule for an electronic cigarette;

FIG. 3b shows a perspective view of a lower housing portion of a capsule for an electronic cigarette;

FIG. 3c shows a perspective view of a lower housing portion of a capsule for an electronic cigarette;

5 FIG. 4a shows a cross sectional view of a heater for an electronic cigarette;

and

FIG. 4b shows a cross sectional view of part of a heater for an electronic cigarette.

### DETAILED DESCRIPTION

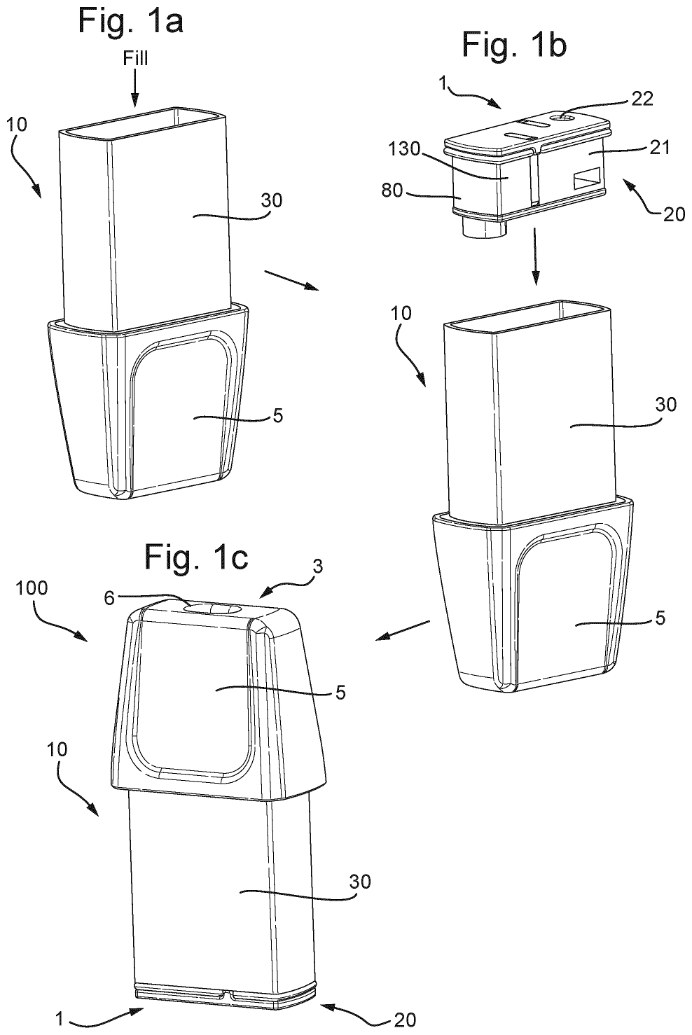

FIG. 1c illustrates a capsule 100 for an electronic cigarette. As most clearly shown in FIG. 1b the capsule 100 comprises an upper housing portion 10 and a lower housing portion 20 which are configured to connect together to form the capsule 100. The capsule has a first end 1 configured to engage with an electronic cigarette device and a second end 3 arranged as a mouthpiece portion 5 having a vapor outlet 6.

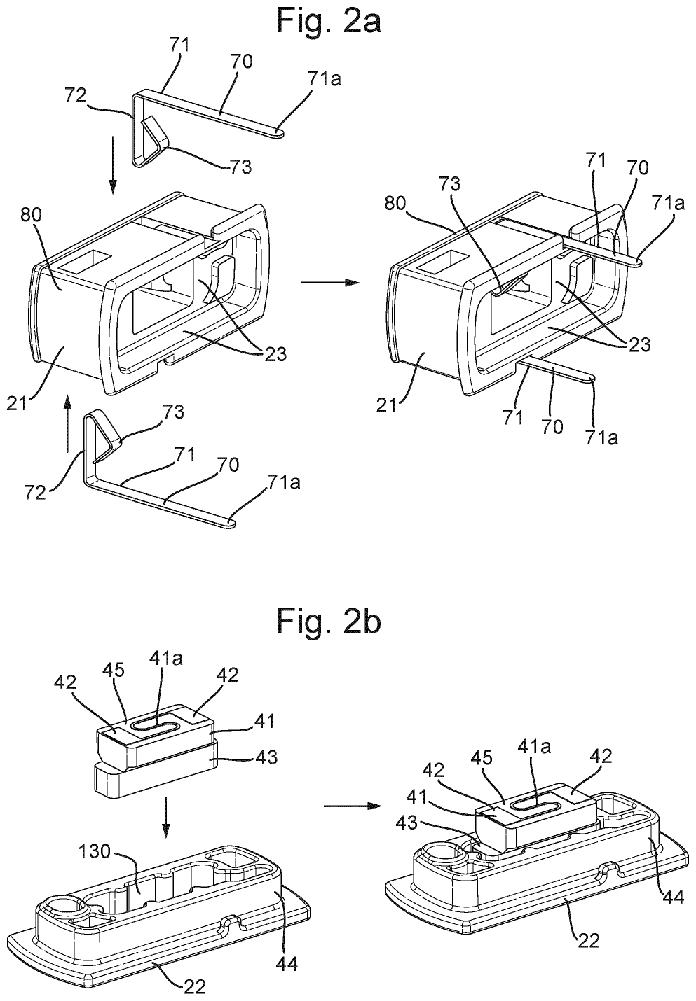

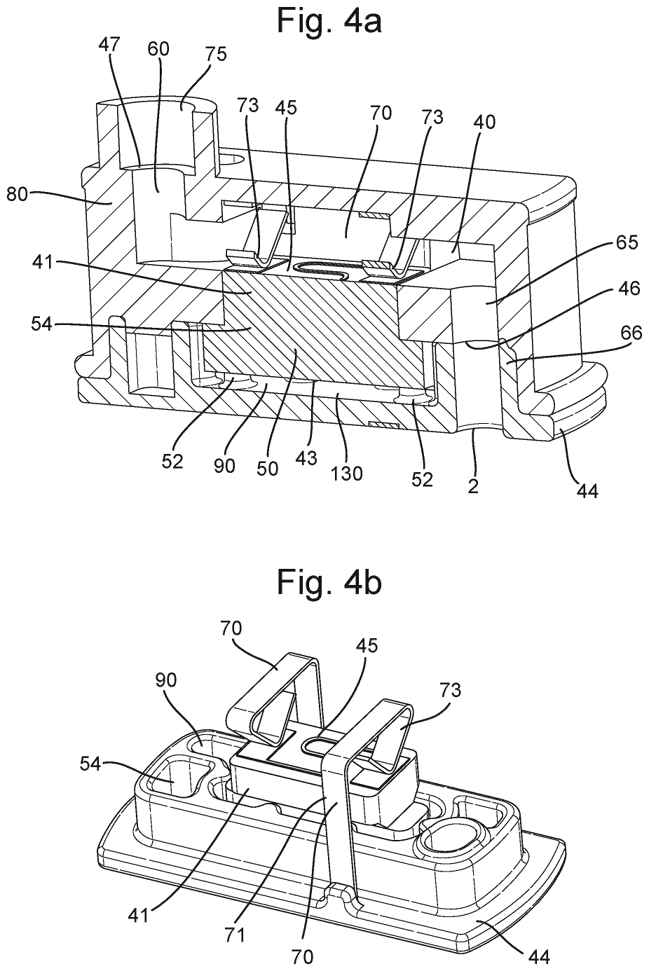

The lower housing portion 20 includes a liquid reservoir 130 arranged to contain a liquid to be vaporised, as shown in FIG. 2b. The liquid reservoir 130 is therefore located towards the first end 1 of the capsule 100. The lower housing portion also includes a vaporising chamber 40, where the vaporising chamber 40 has an air inlet 46 and a vapor outlet 47, illustrated in FIG. 4a. The vaporising chamber 40 is located towards the second end 3 of the capsule 100. A fluid transfer element 50 is positioned between the liquid reservoir 130 and the vaporising chamber 40, and is arranged to transfer liquid between the liquid reservoir 130 and the vaporising chamber 40 by capillary action, as illustrated in

FIG. 4a. A heating element 41 is located between the liquid reservoir 130 and the vaporising chamber 40 and is arranged to heat the liquid that is transferred by capillary action from the liquid reservoir 130 by the fluid transfer element 50. The heating element 41 therefore vaporises the liquid in order to generate a vapor. A flow path 60 extends between the vaporising chamber 40 and the mouthpiece 5 in order to allow the generated vapor to flow from the vaporising chamber 40 to the mouthpiece 5. A portion of the flow path 60 can be seen in FIG. 4a.

The fluid transfer element 50 generally takes the form of a capillary-style wick which is configured to transport liquid from the liquid reservoir 130 through to the vaporising chamber 40 via capillary action through the wick structure, driven by the evaporation of liquid from the centre of the wick by the heating element 41. Generally, the fluid transfer element 50 has an elongate form which extends across the internal volume of the vaporising chamber 40. In this way, when the upper and lower housing portions are brought together as shown in FIG. 1b and the internal volume of the liquid reservoir 130 is filled with liquid, as shown in FIG. 1a, the fluid transfer element 50 is in fluid communication with the liquid within the internal volume of the liquid reservoir 130 and so liquid is drawn into the vaporising chamber 40 through the fluid transfer element 50 during heating.

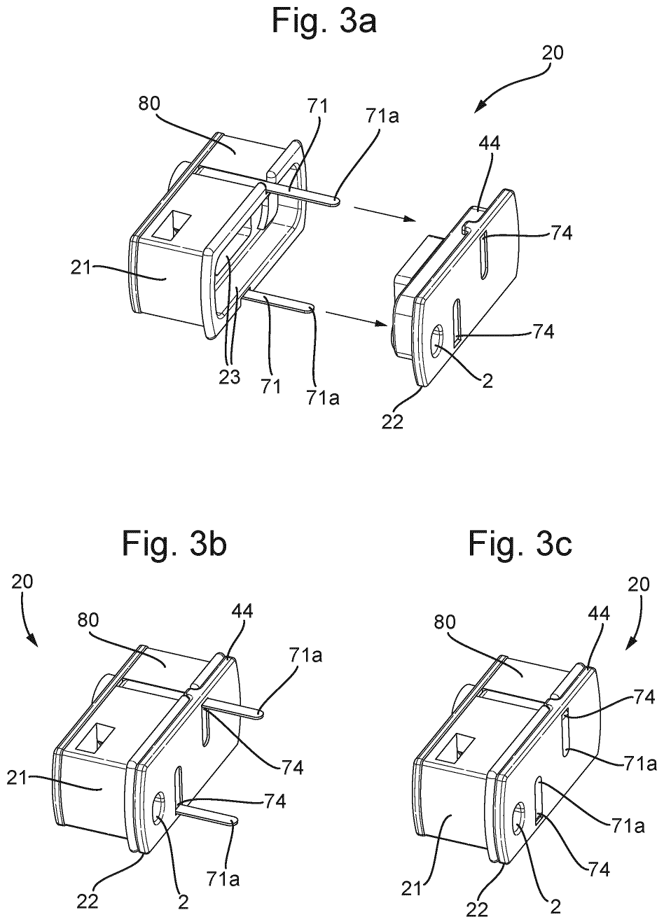

The lower housing portion 20 comprises a seal member 80 and a holder 44, as shown in FIGS. 3a-3c. The seal member 80 has an outer housing wall 21 defining the outer bounds of the lower housing portion 20. As most clearly shown in FIG. 2a the seal member 80 also has a number of internal walls 23 which are arranged to engage with the holder 44.

As can be seen from FIG. 3a, two integral lower housing portions, i.e. the seal member 80 and the holder 44, together form part of the outer housing of the capsule 100 as well as each of the vaporising chamber 40 and liquid reservoir 130. This configuration simplifies the assembly of the capsule because the insertion of separate components within the outer housing, for example to provide the vaporising chamber or the liquid reservoir, is not required. Furthermore the alignment of components, which when not precisely achieved can lead to leakage, can be more accurately achieved by having fewer individual and separately installable components.

As shown, for example, in FIG. 2b, the heating element 41 comprises two contact ends 42 which are arranged to contact first and second electrical contact elements 70. By providing power to the electrical contact elements 70 and subsequently to the heating element 41, the current can be provided through the heating element 41 to heat the heating element 41 and vaporise a liquid transferred from the liquid reservoir 130 through the fluid transport element 50 within the vaporising chamber 40. The heating element 41 is held within the holder 44 which forms the base 22 of the lower housing portion 20.

As can be seen in FIG. 4b, each electrical contact element 70 comprises a longitudinally extending portion 71 which extends substantially parallel to a longitudinal axis of the capsule 100 and a base portion 72 which extends substantially perpendicular to a longitudinal axis of the capsule 100. As can be seen in FIG. 4b, the base portion 72 of each contacting plate 70 comprises a folded region 73 having a substantially triangular shape. The folded region 73 of each electrical contact element 70 is arranged to come into contact with the two ends 42 of the heating element 41.

The electrical contact elements 70 provide the additional function of coupling the seal member 80 to the holder 44 of the lower housing portion 20. As shown in FIGS. 3a and 3b, each longitudinally extending portion 71 passes through a corresponding aperture 74 in the holder 44. The free ends 71a of the longitudinally extending portions 71 are then folded such that they lie substantially flush with an external surface of the base 22, as shown in FIG. 3c. The free ends 71a of the electrical contact elements 70 therefore hold the holder 44 and seal member 80 together to form the lower housing portion 20.

The electrical contact elements 70 are therefore arranged in a substantially U-shaped manner, having a vertically extending portion (i.e. the longitudinally extending portions 71) and two horizontally extending portions (i.e. the base portion 72 and the free ends 71a). It should be noted that vertical and horizontal directions are defined with reference to the capsule when it is held in its operative configuration, as shown in FIG. 1c. Thus, both the base portion 72 and the free ends 71a extend in a direction substantially perpendicularly to the longitudinally extending portion 71. The base portion 72 and the free ends 71a are substantially parallel to each other.

In this way when the capsule 100 is received in an aerosol generating device such as the main body of an electronic cigarette device, the free ends 71a of the electrical contact elements 70 are exposed through the lower housing portion 20, as shown in FIG. 3c, such that they may contact corresponding contacts which are connected to the battery in order to provide current through the contact plate 70 to the heating wire 41.

Further details of the heating element 41 and the liquid reservoir 130 will now be described.

As mentioned, the capsule comprises a fluid pathway 60 which extends from an air inlet 2 of the capsule 100 to the outlet 6 in the mouthpiece 5. The fluid pathway 60 comprises an airflow path 65, a vaporisation flow path 70, and a vapor flow path 75, as shown in FIG. 4a. The airflow path 65 extends through the holder 44 between the air inlet 2 of the capsule 100 and the inlet 46 of the vaporising chamber 40, in order to allow air to enter the vaporising chamber 40. The vaporisation flow path 70 extends through the vaporising chamber 40 between the inlet 46 and the vapor outlet 47 of the vaporising chamber 40. The vapor flow path 75 extends through the upper housing portion 10 between the vapor outlet 47 and the mouthpiece 5, in order to allow the generated vapor to flow from the vaporising chamber to the mouthpiece 5.

As shown in FIG. 4a the holder 44 of the lower housing portion comprises a tubular wall 66 extending through the holder 44, which defines the airflow path 65. The airflow path 65 may be thought of as a tubular passageway or conduit aligned with the elongate axis of the capsule 100. In other words, the airflow path 65 is substantially parallel to a longitudinal axis of the capsule 100. The airflow path 65 extends partially into the seal member 80 in order to fluidly connect with the inlet 46 of the vaporising chamber 40.

Similarly the upper housing portion 10 includes a tubular wall which defines the vapor flow path 75 extending between the vaporising chamber 40 and the mouthpiece 5. The vapor flow path 75 may be thought of as a tubular passageway or conduit aligned with the elongate axis of the capsule 100. In other words, the vapor flow path 75 is substantially parallel to a longitudinal axis of the capsule 100.

The vaporisation flow path 70 extends in a direction that is substantially perpendicular to an axial direction (i.e. a longitudinal axis) of the capsule 100. The vaporisation flow path 70 may therefore be thought of as a transversal passageway. This arrangement increases the length of the vaporisation flow path 70 across the heating element 41. The heating element 41 is therefore exposed to a longer vaporisation flow path 70 allowing a more consistent, as well as a greater volume, of vapor to be generated.

As has been mentioned previously, the heating element 41 comprises a capillary type heating element having two ends 42. The heating element 41 includes a capillary surface 43 which is arranged to receive the liquid to be vaporised from the liquid reservoir 130 and a heating surface 45 which is arranged to vaporise the received liquid, as illustrated in FIG. 2b. The capillary surface 43 therefore carries out the function of the previously described fluid transfer element 50.

The capillary surface 43 is arranged opposite to the heating surface 45 of the heating element 41 which is in contact with air flow. As shown in FIG. 4a, the heating surface 45 is arranged between the main body of the heating element 41 and the vaporising chamber 40, while the capillary surface 43 is arranged between the main body of the heating element 41 and the liquid reservoir 130.

The heating surface 45 of the heating element 41 essentially extends in transversal direction and the air flow through the vaporisation flow path 70 flows in the same direction. Moreover, the heating element is sealed between the capillary surface 43 and the heating surface 45 along its sides by the seal member 80. This means that, by capillary action, the liquid is drawn through the liquid capillary part 43 to the heating surface 45. Liquid is prevented from leaking along the side the heating element 41. Moreover, the liquid gradient at the heating surface 45 in contact with the air is minimized. Thus, the alignment of the heating surface 45 with the air flow is optimised.

In order to aid transfer of the liquid between the liquid reservoir 130 and the heating surface 45, the heating surface 45 and the capillary surface 43 are in fluid communication with each other. To facilitate the transfer, the capillary surface 43 and the heating element 41 are formed from a rigid, porous ceramic, which transfers the liquid from the liquid reservoir 130 via capillary action through the porous structure, driven by the evaporation of liquid by the heating element 41.

A heater track 41a is positioned on the heating surface 45, between the two ends 42 of the heating element 41. The heater track 41a vaporises the received liquid which causes the liquid vapor to be generated within the vaporising chamber 40, which then flows along the vaporisation flow path 70 and out of the vaporising chamber 40. The presence of a heater track 41a may ensure that only the heater track 41a of the heating surface 45 is able to vaporise the received liquid, rather than the whole heating surface 45, which provides more controlled vaporisation of the received liquid. The heater track 41a is arranged to cover enough surface area of the heating surface 45 to provide even heat distribution over the whole heating surface 45. There is typically sufficient heat energy to vaporize the liquid next to the heating track 45 without requiring heating track 41a to be arranged over the entire heating surface 45.

The liquid reservoir 130 includes a buffer reservoir 90 located within the lower housing portion 20 and arranged to store a volume of liquid for vaporisation. Additionally, the capsule comprises a storage reservoir 30 arranged to store an additional volume of liquid for vaporisation. A liquid conduit provides a fluid connection between the buffer reservoir 90 and the storage reservoir 30. The storage reservoir 30 is located between the mouthpiece 5 and the heating element 41, as shown in FIG. 1c. The storage reservoir 30 may therefore be considered as being located within the upper housing portion 10. As such, the storage reservoir 30 is located closer to the mouthpiece 5 than the buffer reservoir 90. This means that when the capsule is held vertically in its operative configuration, as showing in FIG. 1c, the storage reservoir 30 is located above the buffer reservoir 90. This arrangement allows liquid to flow from the storage reservoir 30 through the liquid conduit to the buffer reservoir 90 under the action of gravity.

The buffer reservoir 90 is formed such that it is able to store a substantially constant volume of liquid along the length of the buffer reservoir 90, wherein the length of the buffer reservoir 90 is in a direction that is perpendicular to a longitudinal axis of the capsule 100. In order that the liquid in the buffer reservoir 90 can be vaporised by the heating element 41, the buffer reservoir 90 is located adjacent to the heating element 41. Specifically, the buffer reservoir 41 is adjacent to the capillary surface 43 of the heating element 41 so that the liquid in the buffer reservoir 90 is drawn through the liquid capillary part 43 to the heating surface 45, by capillary action. As mentioned, the heating element 41 extends in transversal direction and so the length of the buffer reservoir 90 is parallel to the length of the heating element 41.

To improve the transfer of liquid from the buffer reservoir 90 to the heating surface 45, a portion of the heating element 41 is located within the buffer reservoir 90. The heating element 41, in particular the capillary surface 45, is supported in the buffer reservoir 90 by a number of spacers 52, two of which can be seen in FIG. 4a. The spacers 52 ensure that the heating element 41 is spaced apart from the base, or floor, of the buffer reservoir 90 so that liquid can flow around the spacers 52 and around the heating element 41, in particular around the capillary surface 45. The spacers 52 form part of the heating element 41 and are located adjacent the capillary surface 45. In some examples, the spacers 52 take the form of a rib. However, in other examples the spacers 52 take the form of a bump.

The spacers 52 therefore provide a support function and are constructed so that liquid can flow between the spacers 52. In particular, the spacers are there to support the heating element 41 and maintain a controlled volume to the heating element 41 as liquid fills the reservoir 90 between the spacers 52. The spacers 52 can therefore be thought of as forming part of the wall of the buffer reservoir 90, the wall being located substantially opposite to the heating element 41.

As can be seen in FIG. 4a, the heating element 41 is located between the holder 44 and the seal member 80. The seal member 80 forms part of the vaporising chamber 40, the air inlet 46, and the vapor outlet 47. These components are therefore integrally formed with the seal member 80, reducing the complexity of the capsule 100. By using a single component (i.e. the seal member 80) rather than a number of individually formed components which need to be connected together, the chance of liquid leaking, for example through joins between the components, is reduced.

Together with the holder 44, the seal member 80 delimits the buffer reservoir 90, in particular an upper boundary of the buffer reservoir 90, as can be seen in FIGS. 4a and 4b. The buffer reservoir 90 is therefore separated from the vaporising chamber 40 by the heating element 41 and the seal member 80. The seal member 80 prevents liquid from leaking from the buffer reservoir 90 into the vaporising chamber 40.

In addition, the heating surface 43 of the heating element 41 can be thought of as forming part of a surface of the seal member 80, as showing in FIG. 4a. As such, the heating surface 45 of the heating element 41 delimits the vaporizing chamber 40, in particular the lower boundary of the vaporizing chamber 40. The heating surface 45 therefore forms a fluid seal with the seal member 80, preventing the flow of vapor and liquid from the vaporizing chamber 40 to the heating element 41.

The capsule, more particularly the holder 44 comprises a liquid collector 54, as most clearly shown in FIG. 4b. The collector 54 collects condensate and liquid which has not been vaporised. As can be seen in FIG. 4a, the liquid collector 54 is positioned substantially opposite the main vapor conduit 75 to collect condensate and prevent it from going back to the vaporizing chamber 40 and leaking into the airflow. The liquid collector 54 is closed so fluid that has been collect in the liquid collector remains in the liquid collector 54 and no recycling of fluid is possible, nor desired, as this may cause leakage.

As previously discussed with reference to FIG. 4b, each electrical contact element 70 comprises a folded region 73. As shown more clearly in FIG. 4a, these folded regions 73 are located within the vaporising chamber 40 and are arranged to contact the ends 42 of the heating element 41. As well as providing an electrical connection, the folded regions 73 hold the heating element 41 within the buffer reservoir 90. The folded regions 73 may therefore be referred to as a gripping mechanism 73 arranged to maintain the position of the heating element 41 within the buffer reservoir 90. The electrical contact elements 70 are therefore able to provide the additional function of ensuring that the heating element 41 is held in the correct position relative to the buffer reservoir 90.

Looking at FIG. 4b, the electrical contact elements 70 are formed from a planar length of metal which has been bent to form the shape of the electrical contact elements 70. In some cases, the electrical contact elements 70 are formed from at least one spring blade. During formation, the folded region 73 is formed such that there is a slight bias. The bias is towards the longitudinally extending portions 71 of the electric contact elements 70, such that the angle between the longitudinally extending portions 71 and the folded region 73 is less than 90 degrees. When the capsule is constructed, as show in FIG. 1c, and partly shown in FIG. 4b, the heating element 41 forces the folded regions 73 to move slightly, creating an angle of substantially 90 degrees with the longitudinally extending portion 71 of the electric contact elements 70. Thus, the bias is in a downward direction, towards the holder 44. In other words, the folded regions 73 are biased such that when they are in contact with the heating element 41, they push down on the heating element 41 towards the holder 44. Specifically, the gripping mechanism 73 engages the heating surface 45 of the heating element 41. The biasing effect of the gripping mechanism 73 helps maintain the position of the heating element 41 within the holder, in particular within the buffer reservoir 90. The gripping mechanism 73 is resiliently biased, meaning that it will return to its original configuration if the heating element 41 were to be removed from the holder 44.

In some cases, the electric contact elements 70, in particular the folded region 73, can be used as a heat shield, to protect the seal member 80 from heat generated by the heating element 41.

As the skilled person will appreciate, the capsule described above, and any of its modifications, can be used as part of an electronic cigarette. For example, an electronic cigarette comprises a main body having a power supply, electrical circuitry, and a capsule seating. The capsule seating of the main body is arranged to engage with and electrically connect with the first end 1 of the capsule described above.

1. A capsule for an electronic cigarette, the capsule having a first end configured to engage with an electronic cigarette device and a second end arranged as a mouthpiece portion having a vapor outlet, the capsule further comprising:
a liquid reservoir located towards the first end of the capsule, the liquid reservoir configured to store a liquid to be vaporised;
a vaporising chamber located towards the second end of the capsule, the vaporising chamber comprising an air inlet and a vapor outlet;
a heating element arranged between the liquid reservoir and the vaporising chamber, the heating element configured to vaporise liquid received from the liquid reservoir and generate a vapor in the vaporising chamber;
a flow path extending between the vaporising chamber and the mouthpiece to allow the generated vapor to flow from the vaporising chamber to the mouthpiece;
wherein the heating element comprises:
a heating surface at a first end of the heating element, the heating surface exposed to the vaporising chamber; and
a capillary element comprising a capillary surface, the capillary surface exposed to the liquid reservoir;
wherein the heating surface is substantially planar; and
wherein the capillary surface is located at a second end of the heating element, and the capillary surface extends at least partially parallel to the heating surface.

2. The capsule according to claim 1, wherein the heating element comprises a capillary-type heating element.

3. The capsule according to claim 1, wherein the heating surface of the heating element comprises a porous material.

4. The capsule according to claim 1, wherein the capillary surface of the heating element comprises a porous material.

5. The capsule according to claim 1, wherein the heating element is made of a porous material, the porous material extending between the capillary surface and the heating surface.

6. The capsule according to claim 3, wherein the porous material comprises a rigid ceramic.

7. The capsule according to claim 1, wherein the heating surface is arranged between a main body of the heating element and the vaporising chamber, and
wherein the capillary surface is arranged between the main body of the heating element and the liquid reservoir.

8. The capsule according to claim 1, wherein the heating surface and the capillary surface are in fluid communication with each other.

9. The capsule of claim 1, wherein the heating surface comprises an electrically resistive surface configured to vaporise liquid received from the liquid reservoir and to generate a vapor in the vaporising chamber.

10. The capsule of claim 9, wherein the electrically resistive surface is directly attached to the heating surface.

11. The capsule according to claim 1, wherein the liquid reservoir comprises a buffer reservoir which is in fluid communication with a storage reservoir via a conduit.

12. The capsule according to claim 11, wherein the storage reservoir is located between the second end of the capsule and the heating element.

13. The capsule according to claim 11, wherein the storage reservoir is located closer to the mouthpiece than the buffer reservoir so that the storage reservoir is located above the buffer reservoir when the capsule is held vertically in its an operative configuration thereof such that liquid can flow from the storage reservoir to the buffer reservoir via the conduit under the action of gravity.

14. The capsule according to claim 11, wherein at least part of the heating element is located within the buffer reservoir.

15. The capsule according to claim 14, wherein the heating element comprises at least one gripping mechanism arranged to maintain a position of the heating element within the buffer reservoir.

16. The capsule according to claim 11, wherein the buffer reservoir comprises a substantially constant volume of liquid along a length of the buffer reservoir, wherein the length of the buffer reservoir is adjacent at least one surface of the heating element.

17. The capsule according to claim 11, wherein the buffer reservoir is delimited by a holder of the capsule comprising a sump and a seal member.

18. An electronic cigarette comprising a main body and a capsule wherein the main body comprises a power supply unit, electrical circuitry, and a capsule seating configured to connect with the capsule, the capsule comprising:
a first end configured to engage with the electronic cigarette device and a second end arranged as a mouthpiece portion having a vapor outlet, the capsule further comprising:
a liquid reservoir located towards the first end of the capsule, the liquid reservoir configured to store a liquid to be vaporised;
a vaporising chamber located towards the second end of the capsule, the vaporising chamber comprising an air inlet and a vapor outlet;
a heating element arranged between the liquid reservoir and the vaporising chamber, the heating element configured to vaporise liquid received from the liquid reservoir and generate a vapor;
a flow path extending between the vaporising chamber and the mouthpiece to allow the generated vapor to flow from the vaporising chamber to the mouthpiece;
wherein the heating element comprises:
a heating surface at a first end of the heating element, the heating surface exposed to the vaporising chamber; and
a capillary element comprising a capillary surface, the capillary surface exposed to the liquid reservoir;
wherein the heating surface is substantially planar; and
wherein the capillary surface is located at a second end of the heating element, and the capillary surface extends at least partially parallel to the heating surface.

19. The capsule according to claim 12, wherein the storage reservoir is located closer to the mouthpiece than the buffer reservoir so that the storage reservoir is located above the buffer reservoir when the capsule is held vertically in an operative configuration thereof such that liquid can flow from the storage reservoir to the buffer reservoir via the conduit under the action of gravity.
